(19) United States
(12) Patent Application Publication
(10) Pub. No.: US 2010/0240970 A1
(43) Pub. Date: Sep. 23, 2010

(54) METHOD FOR AN IN VIVO MEASUREMENT USING A DEVICE IMPLANTED IN A PATIENT, AND CORRESPONDING DEVICE

(76) Inventors: Arne HENGERER, Mohrendorf (DE); Andreas Kappel, Konigstein (DE); Luis Lasalvia, White Plains, NY (US); Andrea Lichte, Marburg (DE); Thomas Mertelmeier, Erlangen (DE); Sven Meyburg, Erlangen (DE); Christoph Petry, Neuss (DE); Marcus Pfister, Bubenreuth (DE); Martin Requardt, Nurnberg (DE)

Correspondence Address: HARNESS, DICKEY & PIERCE, P.L.C., P.O.BOX 8910, RESTON, VA, 20195, US

(21) Appl. No.: 12/723,940

(22) Filed: Mar. 15, 2010

(30) Foreign Application Priority Data
Mar. 17, 2009 (DE) 10 2009 013 594.4

(51) Int. Cl. A61B 5/145; A61M 31/00

(52) U.S. Cl. 600/309; 604/48

(57) ABSTRACT

An embodiment of the present invention relates to a device, which can be implanted into a patient, for an in vivo measurement. In an embodiment, the device includes a catheter for guiding a sample of the patient town analysis unit in the device. A measurement result is determined in the analysis unit by analyzing the sample with the aid of a reagent. In an embodiment, the device furthermore includes an interchange chamber, with the aid of which the reagent can be removed from the device or can be inserted into the device. The interchange chamber has a connector allowing removal or insertion of the reagent while the device is implanted.

### PRIORITY STATEMENT

The present application hereby claims priority under 35 U.S.C. §119 on German patent application number DE 10 2009 013 594.4 filed Mar. 17, 2009, the entire contents of which are hereby incorporated herein by reference.

### FIELD

At least one embodiment of the present invention generally relates to a method for an in vivo measurement using a device implanted in a patient and/or to a device, which can be implanted into a patient, for an in vivo measurement.

### BACKGROUND

In order to ensure continuous monitoring of the state of the patient, a continuous measurement of certain markers, e.g. biomarkers or tumor markers, in bodily fluids such as blood is advantageous in sick patients, for example in cancer patients, patients with an increased risk of a stroke or an increased risk of a myocardial infarction. By way of example, these markers can also be used for monitoring the concentration of a certain medicament with a narrow therapy range. Moreover, it is advantageous to measure certain physical parameters, such as temperature, on a continuous basis. Such measurements are usually carried out by repeated single measurements, for example with the aid of taking blood. If such measurements, which have to be repeated regularly, are carried out at short time intervals, for example a number of times a day or every hour, this is very uncomfortable for the patient.

### SUMMARY

At least one embodiment of the present invention is directed to a continuous measurement or multiple measurements of, for example, markers or physical parameters, which measurement is perceived as less uncomfortable and less unpleasant by the patient.

According to at least one embodiment of the invention, a method is for an in vivo measurement using a device implanted in a patient, and/or a device, which can be implanted into a patient, for an in vivo measurement. The dependent claims define example embodiments of the invention.

According to at least one embodiment of the present invention, provision is made for a method for an in vivo measurement using a device implanted in a patient. According to at least one embodiment of the method, a sample of the patient is automatically guided to an analysis unit in the device with the aid of an implanted catheter of the device. Analyzing the sample with the aid of a reagent automatically determines a measurement result in the analysis unit. The reagent can be removed from the device or inserted into the device by way of an interchange chamber coupled to the analysis unit while the device is implanted in the patient.

By virtue of the fact that the sample of the patient is automatically guided to the implanted analysis unit with the aid of the implanted catheter and is automatically analyzed in the analysis unit, removal of the sample by, for example, a medical practitioner or the patient is not necessary. By way of example, the analysis can be performed automatically at predetermined time intervals and so continuous monitoring of the patient is made possible. Since the analysis does not require an intervention into the patient, this method is particularly pleasant for the patient.

Many types of analysis require a reagent that is used up during the analysis. By affording the possibility of removing the reagent from the implanted device or inserting the reagent into the implanted device by way of the interchange chamber while the device is implanted in the patient, the reagent can be replaced without uncovering the device with the aid of a complicated operation. Hence, a used reagent can easily be replaced by a new reagent. Moreover, a first reagent for example can be exchanged for a second reagent in order to carry out a different type of analysis and examination.

The interchange chamber can be implanted into the patient as a component of the implanted device. In this case, the reagent is interchanged for example with the aid of a syringe with an injection needle, which penetrates a connector of the interchange chamber through the skin of the patient and interchanges the reagent.

Alternatively, the interchange chamber can be arranged outside of the patient and can be coupled to the analysis unit via a suitable connection passing through the skin of the patient. This affords a more pleasant interchange of the reagent for the patient. By way of example, the interchange chamber can be attached to the skin of the patient in the vicinity of the analysis unit with the aid of a plaster.

According to at least one embodiment of the present invention, provision is made for a further method for an in vivo measurement using a device implanted in a patient. In this method, a first sample of the patient is guided from a first location in the patient to an analysis unit in the device with the aid of a first implanted catheter of the device. Furthermore, a second sample of the patient from a second location in the patient is guided to the analysis unit or to a further analysis unit with the aid of a second implanted catheter. In the analysis unit or the further analysis unit, a first measurement result is determined by analyzing the first sample and a second measurement result is determined by analyzing the second sample. The measurement results are compared with the aid of a processing unit in the implanted device.

By way of example, in order to examine a distribution of a medicament, two samples from different locations in the patient can be examined using this method. By way of example, one examined sample can be from fatty tissue and a blood sample can be a further sample. Using this, the distribution of certain lipophilic medicaments, such as valium, can be examined. The concentration in the blood for example determines the direct effectiveness of the medicament, whereas the concentration in the fatty tissue provides information about the expected long-term effectiveness. By way of example, automatic medicament dispensation can be controlled by a measurement of the two samples. Moreover, different parameters can be measured at different locations in the body. Thus, for example, a tumor marker can be measured in the blood and a temperature can be measured at a certain location in the body. Since the measurement results are automatically determined in the body of the patient, complicated removal procedures are not required and continuous monitoring at regular time intervals can be performed in a simple fashion.

According to an embodiment, the measurement results can be transmitted from the processing unit to a further device outside of the patient. By way of example, the transmission can be carried out wirelessly. The device outside of the patient can be e.g. a computer system, which further processes the data from the processing unit, stores the data or transmits it to a treating medical practitioner for further processing. As a result of this, the measurement results can be used directly for a further treatment of the patient.

According to a further embodiment, the measurement results additionally can be stored electronically in the implanted device. As a result of this, a plurality of measurement results detected over a relatively long period of time first of all can be buffer-stored in the implanted device before they are for example transmitted to the further device outside of the patient in order to be further utilized there.

According to a further embodiment, the guidance of the sample of the patient to the analysis unit can comprise pumping a bodily fluid of the patient with the aid of a pump of the device. The bodily fluid can be e.g. blood from a patient's blood vessel which has the catheter inserted therein. Moreover, the bodily fluid also can comprise e.g. urine, which, for example, is guided to the analysis unit from the bladder of the patient via an appropriate catheter. By pumping the bodily fluid to the analysis unit with the aid of a pump, a predetermined amount of the sample can be reliably guided into the analysis unit and so precise analysis results are obtained.

According to a further embodiment, a warning signal can be produced for the patient, depending on the measurement result or results. By way of example, the warning signal can comprise an acoustic signal, a tactile signal or a radio signal to a receiver worn by the patient. By way of example, the warning signal can be triggered if it was established on the basis of the measurement result that a medicament level, for example in the blood of the patient, has dropped below a prescribed value.

According to a further embodiment, a medicament is automatically dispensed in the patient by the implanted device, depending on the measurement result. For example, if a medicament level in the blood of the patient drops below a prescribed level, this shortfall is determined by analyzing the blood in the implanted analysis unit and automatic medicament dispensation can be initiated by the device. This affords very precise automatic maintenance of a desired medicament level.

According to a further embodiment, analyzing the sample can furthermore comprise determining a pH value, determining a concentration of a biomarker or tumor marker, or determining a concentration of a medicament. Moreover, for example, a temperature of the sample can be determined; or a pressure, a partial pressure or a flow velocity of a bodily fluid can be determined with the aid of suitable sensors on the catheter. By way of example, this can ensure reliable and continuous monitoring of the cardiovascular system of the patient.

According to at least one embodiment of the present invention, provision is furthermore made for a device for an in vivo measurement, which device can be implanted into a patient. The device comprises a catheter and an analysis unit, which is coupled to an interchange chamber. The catheter is designed to guide a sample of the patient to the analysis unit. The analysis unit is designed to determine a measurement result by analyzing the sample with the aid of a reagent. The interchange chamber can be implanted into the patient as part of the device or can be arranged outside of the patient. The interchange chamber is designed such that the reagent can be removed from the interchange chamber or the reagent can be inserted into the interchange chamber while the device, and hence possibly the interchange chamber as well, is implanted in the patient. To this end, the interchange chamber has a connector allowing the removal and insertion of the reagent.

By way of example, the connector can be a membrane, which can be punctured by an injection needle and which automatically again closes off a hole formed during a puncturing by the injection needle after the injection needle has been removed. The interchange chamber can preferably be implanted just under the skin of the patient and so only the skin of the patient and the connector of the interchange chamber have to be punctured by the injection needle in order to insert or remove the reagent. As a result, the implantable device can be used in the patient for a long time because a reagent required for the analysis can be easily interchanged or inserted.

According to at least one embodiment of the present invention, provision is made for a further device for an in vivo measurement, which device can be implanted into a patient. The device comprises a first catheter for guiding a first sample of the patient from a first location in the patient to an analysis unit in the device and a second catheter for guiding a second sample of the patient from a second location in the patient to the analysis unit. The analysis unit is designed to determine a first measurement result by analyzing the first sample and a second measurement result by analyzing the second sample. The device furthermore comprises a processing unit for comparing the measurement results of the analysis unit. By analyzing samples from two different locations in the patient and by being able to process further the results in a processing unit, it is possible, for example, to determine a medicament distribution in the body or to measure a marker concentration, such as a biomarker or tumor marker, at different locations in the body. As a result, changes in the body of the patient can be detected continuously and appropriate warnings possibly can be emitted or information for further treatment can be derived.

According to an embodiment, the above-described devices moreover can also comprise a transmission unit for transmitting the measurement results to a further device outside of the patient. By way of example, this transmission unit can comprise a radio transmission unit designed for wireless transmission of the measurement results. As a result, the measurement results for example can be transmitted as raw data from the analysis unit or the analysis units to the device outside of the patient in order to be further processed there.

According to a further embodiment, the device can comprise a pump for relaying a bodily fluid of the patient to the analysis unit. If the sample is a bodily fluid, such as blood, the use of this pump can guide a defined amount of this bodily fluid to the analysis in the analysis unit. This ensures high precision in the analysis.

The device can furthermore comprise an output unit for emitting a warning signal for the patient. By way of example, the warning signal can be triggered depending on one or more measurement results. By way of example, the warning signal can comprise e.g. an acoustic signal, a tactile signal, e.g. an oscillation of a housing of the processing unit, or a radio signal, which is received by a receiver outside of the body of the patient. As a result, the patient can be informed directly that, for example, a medicament concentration is outside of a permissible concentration range or another monitored value of the body of the patient is not within a predetermined range. Depending on the warning signal, the patient can then, for example, take a medicament or visit a medical practitioner.

According to a further embodiment, the device furthermore comprises a medicament-dispensing unit. The processing unit is coupled to the medicament-dispensing unit and designed such that, depending on the measurement result, it drives the medicament-dispensing unit such that the medicament-dispensing unit dispenses a medicament to the patient. As a result, a predetermined medicament concentration in the body of the patient for example can be monitored and maintained automatically.

According to a further embodiment of the present invention, the device comprises an electronic storage in which the processing unit stores the measurement results. The measurement results then can be recalled for example from the storage by the processing unit at a desired time and can be transmitted to a further device outside of the patient in order to continue to be analyzed there. This for example allows a medical practitioner to recall and examine the profile of e.g. a medicament concentration or a marker concentration over a predetermined recorded period of time from the implanted device.

According to a further embodiment, the analysis unit is designed to determine e.g. a pH value, a concentration of a biomarker or a concentration of a medicament. The analysis unit furthermore can comprise sensors for automatically determining a temperature, a pressure, a partial pressure or a flow velocity at a predetermined location in the body of the patient.

### BRIEF DESCRIPTION OF THE DRAWINGS

In the following text, the present invention will be explained on the basis of example embodiments with reference to the drawings.

FIG. 1 schematically shows an embodiment of a device, which can be implanted into a patient, according to an embodiment of the present invention and a patient with the implanted device.

FIG. 2 shows a further embodiment of a device, which can be implanted into a patient, according to an embodiment of the present invention and a patient with the implanted device.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

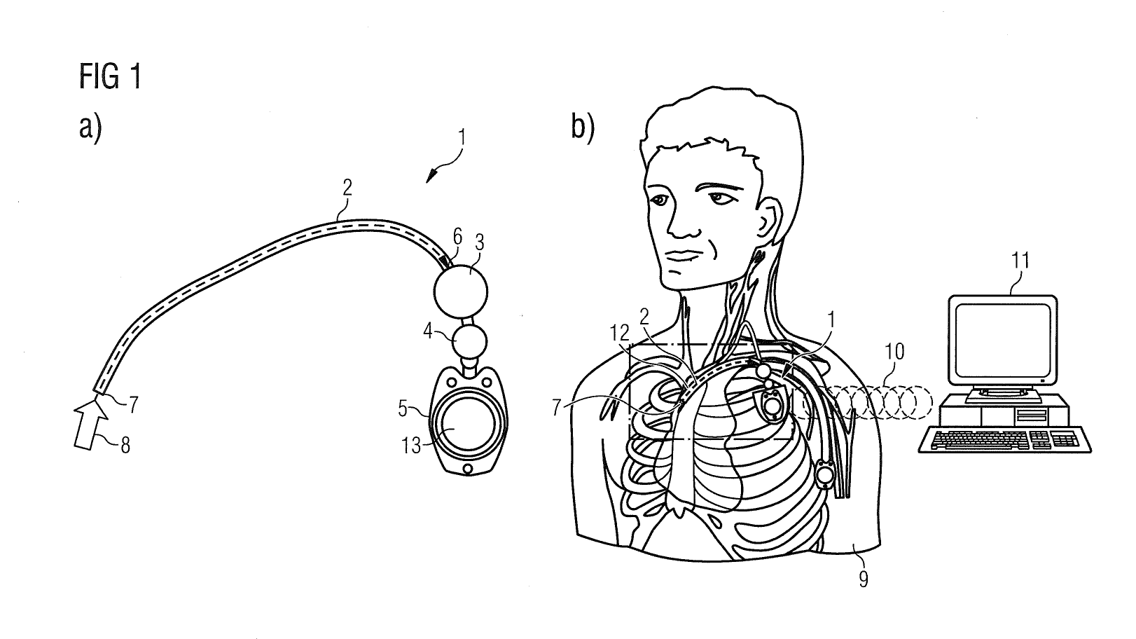

FIG. 1a shows a device 1, which can be implanted into a patient, for an in vivo measurement. The device 1 comprises a catheter 2, an analysis unit 3, a processing unit 4, and an interchange chamber 5. The catheter 2 is connected to the analysis unit 3 at a first end 6 of the catheter 2. A second end 7 of the catheter 2 is open. The analysis unit 3 comprises a pump in order to guide a liquid through the catheter 2 and into the analysis unit 3 in the direction illustrated by the arrow 8. The analysis unit 3 is furthermore connected to the processing unit 4 and the interchange chamber 5.

FIG. 1b shows a patient 9, into whom the implantable device 1 from FIG. 1a is implanted. The catheter 2 is implanted in a blood vessel 12 of the patient 9. The second, open end 7 of the catheter 2 ends in the blood vessel 12. If the pump of the analysis unit 3 is actuated, blood from the blood vessel 12 is guided into the analysis unit 3 via the catheter 2. The pumped-in blood is analyzed in the analysis unit 3 with the aid of a reagent from the interchange chamber 5. A result of the analysis is transmitted from the analysis unit 3 to the processing unit 4, which stores the result and, when necessary, transmits it to an external evaluation device 11, such as a computer system, via a radio connection 10. The processing unit 4 for example can comprise a microprocessor control, which is supplied by a battery in the processing unit 4. By way of example, the processing unit 4 can be programmed by the external evaluation device 11 via the radio connection 10 such that it pumps blood from the blood vessel 12 into the analysis unit 3 at predetermined intervals and analyzes said blood.

Alternatively, the catheter 2 also can be inserted into e.g. the bladder of the patient 9 in order to pump urine samples into the analysis unit 3 and analyze the samples. Furthermore, the second, open end 7 of the catheter 2 can be situated at a location in the patient with soft tissue in order to guide a cell fluid of the soft tissue to the analysis unit 3 and examine it in respect of tumor markers or biomarkers.

The measurement results of the analysis unit 3 can be further processed in the processing unit 4, e.g. in order to trigger an alarm or control a medicament dispensation with the aid of a medicament-dispensing unit (not illustrated). By way of example, the alarm can comprise an acoustic signal, a vibration signal or a radio signal to a receiver worn by the patient 9. By way of example, the alarm can remind the patient to take a medicament or can advise the patient to visit a medical practitioner because e.g. a critical state was determined. The analysis data can also be stored as so-called raw data in the processing unit 4 and can be transmitted to the external evaluation device 11 via the radio connection 10.

In addition to the abovementioned measurements of certain biomarkers and tumor markers, physical parameters, such as a temperature, pressures, a bio-impedance, a pH value, a partial pressure or flows also can be determined and recorded with the aid of suitable sensors coupled to the processing unit 4 and can be transmitted to the external evaluation device 11. In addition to determining the concentration, the analysis unit 3 also can carry out e.g. photometric, turbidimetric, impedance or PCR analyses.

The interchange chamber 5 comprises a reagent, which is used for the analysis in the analysis unit 3. So that the reagent can be interchanged or refilled in order to carry out more analyses, the interchange chamber 5 has a connector 13. In the illustrated embodiment, the connector 13 comprises a membrane located just below the skin of the patient 9 in the implanted state of the interchange chamber 5. The membrane 13 is designed such that it can easily be punctured by an injection needle. In order to interchange the reagent in the interchange chamber 5, an injection needle, for example, can puncture the skin above the membrane 13 and puncture the membrane 13; the used reagent can be suctioned-off through the injection needle with the aid of a syringe and a new reagent can be inserted though the injection needle. Moreover, the membrane 13 is designed such that a hole formed during puncturing by the injection needle automatically closes itself off after the injection needle has been removed and so the interchange chamber 5 again is sealed in respect of the patient 9.

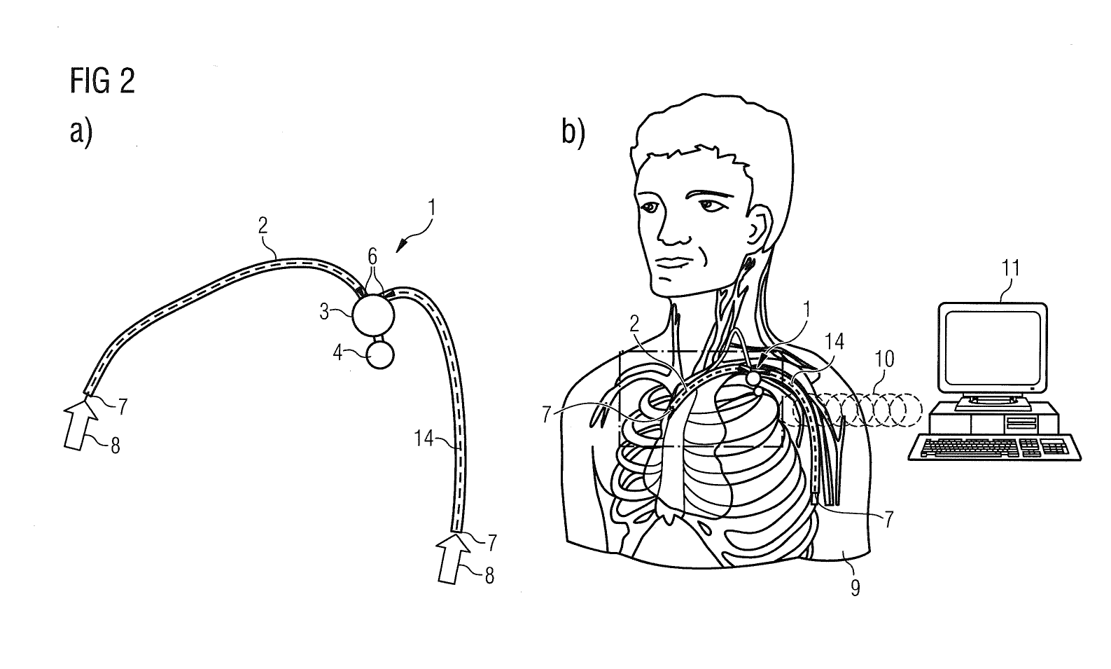

FIG. 2a shows a further embodiment of a device 1, which can be implanted into a patient, for an in vivo measurement. The device 1 comprises two catheters 2, 14, an analysis unit 3, and a processing unit 4. The two catheters 2, 14 respectively have a first end 6, which is connected to the analysis unit 3, and respectively have a second end 7, which is open. The analysis unit 3 is coupled to the processing unit 4. The analysis unit 3 comprises a pump, which selectively suctions a liquid through one of the two catheters 2, 14 into the analysis unit 3 in the direction illustrated by the arrow 8. The suctioned liquid is analyzed in the analysis unit 3 and a corresponding measurement result is determined with the aid of the processing unit 4. By way of example, the processing unit 4 can comprise a microprocessor control. Alternatively, the device 1 can also comprise two analysis units 3, which are each coupled to one of the catheters 2 or 14. In this case, both analysis units 3 are connected to a processing unit 4. Each of the analysis units 3 respectively analyzes a liquid suctioned in through the corresponding catheter 2 or 14, and the processing unit 4 determines corresponding measurement results according to both analyses of the two analysis units 3.

FIG. 2b shows a patient 9, into whom the device 1 from FIG. 2a is implanted. As illustrated in FIG. 2b, the two open, second ends 7 of the catheters 2 and 14 are located at different locations in the patient 9. Hence, analyses for two different locations in the body of the patient 9 are carried out in the analysis unit 3 and further processed with the aid of the processing unit 4. Thus, for example, a parameter can be determined at different locations in the body, for example a medicament concentration in the blood and in a fatty tissue, and it can be compared. Alternatively, different parameters from different locations in the body, e.g. a tumor marker in the blood and a local temperature at a specific location in the body, also can be measured and can be compared. The measurement values for example can be transmitted via a radio connection 10 to an external evaluation device 11 and can be further processed there. Alternatively, the processing unit 4 can also trigger an alarm on the basis of the measurement values, for example an acoustic alarm or a vibration alarm to make the patient aware of an unusual or critical state and to request that the patient take a medicament.

Moreover, the features of the two embodiments of the present invention described above can be combined with one another. Thus, for example, the embodiment illustrated in FIG. 2a can be extended by one or more interchange chambers 5 and so a reagent or a plurality of reagents for the analysis can be interchanged in the single analysis unit or in the plurality of analysis units.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for an in vivo measurement using a device implanted in a patient, comprising:
automatically guiding a sample of the patient to an analysis unit in the device with the aid of an implanted catheter of the device;
automatically determining a measurement result by analyzing the sample with the aid of a reagent in the analysis unit and transmitting the measurement result to a processing unit in the device; and
removing the reagent from an interchange chamber coupled to the analysis unit or inserting the reagent into the interchange chamber while the device is implanted in the patient.

2. The method as claimed in claim 1, wherein the interchange chamber, as a component of the implanted device, is implanted into the patient.

3. The method as claimed in claim 1, wherein the interchange chamber is arranged outside of the patient.

4. A method for an in vivo measurement using a device implanted in a patient, comprising:
automatically guiding a first sample of the patient from a first location in the patient to an analysis unit in the device with the aid of a first implanted catheter of the device;
automatically guiding a second sample of the patient from a second location in the patient to the analysis unit with the aid of a second implanted catheter of the device;
automatically determining a first measurement result by analyzing the first sample and a second measurement result by analyzing the second sample in the analysis unit; and
comparing the first and second measurement results with the aid of a processing unit in the device.

5. The method as claimed in claim 1, wherein the measurement result is transmitted from the processing unit to a further device outside of the patient.

6. The method as claimed in claim 5, wherein the transmission is carried out wirelessly.

7. The method as claimed in claim 1, wherein the guiding of the sample of the patient to the analysis unit comprises pumping a bodily fluid of the patient with the aid of a pump of the device.

8. The method as claimed in claim 1, wherein a warning signal is produced for the patient, depending on the measurement result.

9. The method as claimed in claim 8, wherein the warning signal comprises an acoustic signal, a tactile signal or a radio signal.

10. The method as claimed in claim 1, wherein a medicament is automatically dispensed to the patient by the device, depending on the measurement result.

11. The method as claimed in claim 1, wherein the measurement result is stored electronically in the device.

12. The method as claimed in claim 1, wherein the analyzing of the sample comprises determining a pH value, determining a concentration of a biomarker, determining a concentration of a tumor marker or determining a concentration of a medicament.

13. The method as claimed in claim 1, wherein the analyzing of the sample comprises determining a temperature, a pressure, a partial pressure or a flow velocity.

14. A device, which can be implanted into a patient, for an in vivo measurement, comprising:
a catheter to guide a sample of the patient to an analysis unit in the device; and
the analysis unit to determine a measurement result by analyzing the sample with the aid of a reagent, the analysis unit being coupled to an interchange chamber for removing the reagent from the device or for inserting the reagent into the device, the interchange chamber including a connector to allow a removal or insertion of the reagent while the device is implanted into the patient.

15. The device as claimed in claim 14, wherein the interchange chamber, as a component of the implantable device, is implantable into the patient.

16. The device as claimed in claim 14, wherein the interchange chamber is attachable outside of the patient.

17. The device as claimed in claim 14, wherein the connector comprises a membrane, which is puncturable by an injection needle and which automatically closes off a hole formed during a puncturing by the injection needle after the injection needle has been removed.

18. A device, implantable into a patient, for an in vivo measurement, comprising:
a first catheter to guide a first sample of the patient from a first location in the patient to an analysis unit in the device;
a second catheter to guide a second sample of the patient from a second location in the patient to the analysis unit;
the analysis unit to determine a first measurement result by analyzing the first sample and a second measurement result by analyzing the second sample; and
a processing unit to compare the first and second measurement results.

19. The device as claimed in claim 14, further comprising:
a transmission unit to transmit the measurement result to a further device outside of the patient.

20. The device as claimed in claim 19, wherein the transmission unit comprises a radio transmission unit for the wireless transmission of the measurement result or results.

21. The device as claimed in claim 14, further comprising a pump to guide a bodily fluid as a sample of the patient to the analysis unit.

22. The device as claimed in claim 14, further comprising an output unit to emit a warning signal for the patient, depending on the measurement result.

23. The device as claimed in claim 22, wherein the output unit is designed to emit an acoustic signal, a tactile signal or a radio signal.

24. The device as claimed in claim 14, further comprising:
a medicament-dispensing unit, wherein the processing unit is designed to, depending on the measurement result, drive the medicament-dispensing unit such that the medicament-dispensing unit dispenses a medicament to the patient.

25. The device as claimed in claim 14, further comprising:
an electronic storage, wherein the processing unit is designed to store the measurement result in the electronic storage.

26. The device as claimed in claim 14, wherein the analysis unit is designed to determine a pH value, a concentration of a biomarker, a concentration of a tumor marker or a concentration of a medicament in the sample.

27. The device as claimed in claim 14, wherein the analysis unit is designed to determine a temperature, a pressure, a partial pressure or a flow velocity.

28. The method as claimed in claim 4, wherein the first and second measurement results are transmitted from the processing unit to a further device outside of the patient.

29. The method as claimed in claim 4, wherein a warning signal is produced for the patient, depending on the first and second measurement results.

30. The method as claimed in claim 4, wherein a medicament is automatically dispensed to the patient by the device, depending on the first and second measurement results.

31. The device as claimed in claim 18, further comprising:
a transmission unit to transmit the first and second measurement results to a further device outside of the patient.

32. The device as claimed in claim 18, further comprising an output unit to emit a warning signal for the patient, depending on the first and second measurement results.

33. The device as claimed in claim 18, further comprising:
a medicament-dispensing unit, wherein the processing unit is designed to, depending on the first and second measurement results, drive the medicament-dispensing unit such that the medicament-dispensing unit dispenses a medicament to the patient.
